(19) United States
(12) Patent Application Publication
(10) Pub. No.: US 2019/0178834 A1
(43) Pub. Date: Jun. 13, 2019

(54) Bead Mixer / Cleaner For Use With Sensor Devices

(71) Applicant: Thermo Orion Inc., Chelmsford, MA (US)

(72) Inventors: Julie Gargas-Mozzer, West Newbury, MA (US); Gang Wang, Arlington, MA (US); Ugljesa Krstanovic, Arlington, MA (US); Xiaowen Wen, Lexington, MA (US)

(21) Appl. No.: 16/209,041

(22) Filed: Dec. 4, 2018

Related U.S. Application Data
(60) Provisional application No. 62/597,625, filed on Dec. 12, 2017.

(51) Int. Cl. G01N 27/38; B01F 5/10; G01N 27/08; G01N 33/18; B01L 3/00; G01N 1/38

(52) U.S. Cl. CPC G01N 27/38; B01F 5/104; G01N 1/38; G01N 33/1886; B01L 3/502; G01N 27/08

(57) ABSTRACT

A self-cleaning analyzer system for sensing a chemical characteristic of a fluid sample according to one embodiment includes a sensing chamber including a sample inlet configured to receive the fluid sample and a sample outlet; a sensor configured to sense the chemical characteristic of the fluid sample in the sensing chamber; a plurality of cleaning beads contained in the sensing chamber; and an agitator configured to stir the fluid sample in the sensing chamber. One or more of the plurality of cleaning beads contact the sensor when the agitator stirs the fluid sample. A method for sensing a chemical characteristic of a fluid sample using a self-cleaning analyzer system according to one embodiment includes providing the fluid sample to the sensing chamber; sensing the chemical characteristic of the fluid sample; and stirring the fluid sample causing one or more of the plurality of cleaning beads to contact the sensor.

The present application claims the filing benefit of U.S. Provisional Application Ser. No. 62/597,625, filed Dec. 12, 2017, the disclosure of which is incorporated herein by reference in its entirety.

### FIELD OF THE INVENTION

The present invention generally relates to a bead mixer/cleaner for use with potentiometric sensor devices, such as ion specific electrodes (ISE) and Redox sensor devices, and methods for using same and, more particularly, to a self-cleaning analyzer system including a bead mixer.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light and not as admissions of prior art.

Processes in many industries include a treatment step for waste water generated during the process. For instance, cooling circuits in industrial plants often employ water prone to biofouling. In other industrial settings, such as in large-scale shipping operations, the amount of organic material allowed to exist in waste water, or ballast water, is typically limited by various applicable regulations. As a result, various water treatment protocols are known.

Typical water treatment protocols involve the addition of chlorinated compounds, such as sodium hypochlorite and chlorine dioxide, to the water to disinfect any biological material present in the water. Although such a chlorine treatment is effective at mitigating the effects of biological materials, overuse or underuse of the chlorinated compound can lead to additional problems.

For instance, costs of treatment are greatly increased when too much chlorinated compound is used. Additionally, the outflow of oxidant compounds from industrial processes is often regulated by governing bodies that set an upper limit on the amount of oxidants allowed in the outflow. On the other hand, if too little chlorinated compound is used, the treatment may be ineffective, leading to fouling of the process apparatus or non-compliance with the applicable regulations regarding outflow of biological materials.

As a result, many industries rely on the rapid and accurate measurement of the amount of residual oxidizing material remaining in a sample of water. In fresh water, measurement of the amount of chlorine in the sample is referred to as the Total Residual Chlorine concentration (hereinafter “TRC”), and in sea water, the same measurement is referred to as Total Residual Oxidant concentration (hereinafter “TRO”), owing to the presence of iodide and bromide ions in sea water. Applications as diverse as shipping vessels, water treatment plants, manufacturing centers, thermoelectric and nuclear power stations, oil extraction apparatuses, chemical plants, food production facilities, water pipelines, or any other application in which water is used for manipulating the local environment, all rely on rapid and accurate measurement of residual oxidizing material remaining in the water.

For example, the shipping industry is subject to many regulations, e.g., from the U.S. Environmental Protection Agency, regarding the purity of the water expelled from ballast water tanks, regarding both un-neutralized organic materials and excess chlorinated compounds. In general, when a shipping vessel discharges its cargo at one port, it loads one or more ballast tanks with water adjacent to its hull to help stabilize the vessel. The water that is taken on remains in the ballast tanks until the ship arrives at the next port to take on cargo. As the cargo is loaded, the ballast tanks are emptied through ballast pipes or ducts, either partially or fully, because the ballast water is no longer necessary due to the added weight of the cargo. Because the ship will travel great distances between the two ports, current regulations require biocidal treatment of the water held in the ballast tanks, prior to the ballast water being discharged, to help prevent the proliferation of non-native species of organisms. Practical matters require a similar treatment protocol to remove biological material capable of leading to biofouling of the tanks. The treated water in the ballast tanks should be monitored to control the amount of chlorine added and to ensure that enough chlorine is added to treat the ballast water effectively. Analogously, the applications listed above also require monitoring of the oxidant materials in the outflow of those applications.

TRO readings are subject to interference from other chemicals or particles that may be found in the waste water. In that regard, TRO probe fouling often causes TRO readings to decrease even when the true TRO level remains unchanged. This may cause a controller operating at a predetermined target TRO level to unnecessarily continue to feed chlorine based on the inaccurate measurement.

It is well established that TRO probes must be cleaned to maintain the accuracy of the measurements. However, manually cleaning the TRO probe surfaces is often not practical, especially when the probe is integrated in on-line or in-line analyzer systems. Automatic cleaning systems are used to reduce the cleaning requirements for TRO probes. However, these TRO probe cleaning systems are often complicated and expensive to operate.

Therefore, there is a need for a simple, cost effective method for cleaning an analyzer system for sensing a chemical characteristic of a fluid sample that enables continued accurate measurements.

### SUMMARY OF THE INVENTION

Certain exemplary aspects of the present invention are set forth below. It should be understood that these aspects are presented merely to provide the reader with a brief summary of certain forms the present invention might take and that these aspects are not intended to limit the scope of the present invention. Indeed, the present invention may encompass a variety of aspects that may not be explicitly set forth below.

In accordance with the principles of the present invention, and in the exemplary environment of a shipping vessel dumping ballast water into the proximate environment, a self-cleaning analyzer system according to one embodiment of the present invention may be installed after construction of the shipping vessel in many instances and may maintain accurate measurements of the chemical characteristic of a fluid sample during use due to the self-cleaning aspect. The analyzer system may be placed as close as possible to the ballast water outlet to ensure the highest quality measurement of the concentration of oxidant species at the location of its highest likelihood of environmental impact.

According to one aspect of the present invention, a self-cleaning analyzer system is provided for sensing a chemical characteristic of a fluid sample. The system includes a sample inlet configured to receive the fluid sample and a sample outlet; a sensor configured to sense the chemical characteristic of the fluid sample in the sensing chamber; a plurality of cleaning beads contained in the sensing chamber; and an agitator configured to stir the fluid sample and the plurality of cleaning beads in the sensing chamber. One or more of the plurality of cleaning beads contact the sensor when the agitator stirs the fluid sample.

In another aspect of the present invention, a method is provided for sensing a chemical characteristic of a fluid sample using a self-cleaning analyzer system. The method includes providing the fluid sample to the sensing chamber; sensing the chemical characteristic of the fluid sample; and stirring the fluid sample and cleaning beads in the sensing chamber, thereby causing one or more of the plurality of cleaning beads to contact the sensor.

The above and other objects and advantages of the present invention shall be made apparent from the accompanying drawings and the description thereof.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention

FIG. 1 is a diagrammatic view of an exemplary self-cleaning analyzer system of the present invention shown installed in a ballast discharge duct of a shipping vessel.

FIG. 2 is a diagrammatic cross-sectional view of a self-cleaning analyzer system for sensing a chemical characteristic of a fluid sample according to one aspect of the present invention.

FIG. 3 is a diagrammatic cross-sectional view of a self-cleaning analyzer system for sensing a chemical characteristic of a fluid sample according to another aspect of the present invention.

FIG. 4 is a diagrammatic cross-sectional view of a self-cleaning analyzer system for sensing a chemical characteristic of a fluid sample according to another aspect of the present invention.

### DETAILED DESCRIPTION OF THE INVENTION

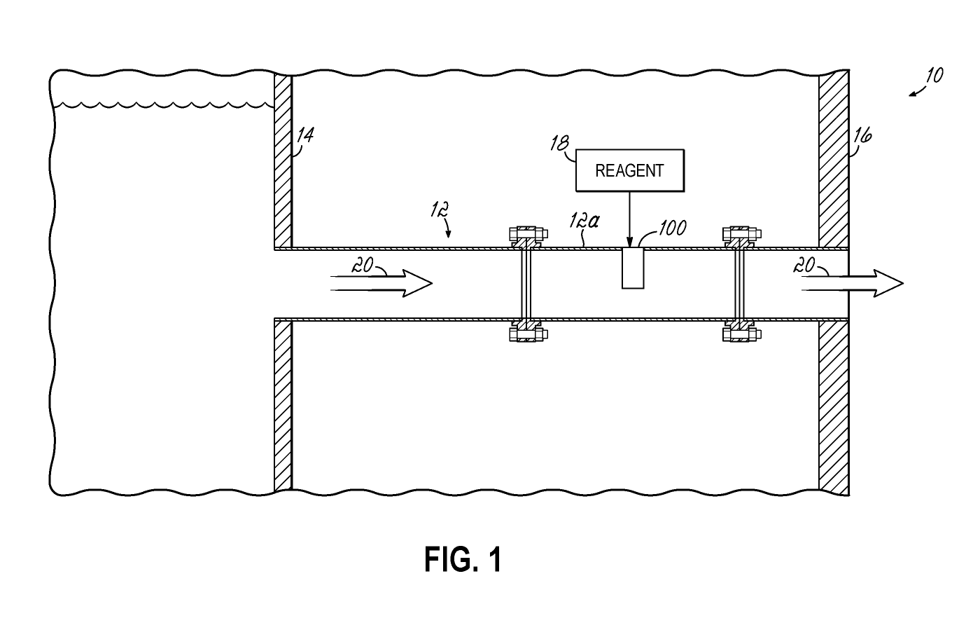

Embodiments of the present invention are directed to self-cleaning analyzer systems that include a sensor, such as potentiometric sensor devices (e.g., ion specific electrodes (ISE) and Redox sensor devices). Referring now to the figures in which like numerals represent like parts, and to FIG. 1 in particular, an analyzer system 100 according to one embodiment of the present invention is shown installed in a shipping vessel 10. Specifically, the system 100 is installed in a ballast water discharge duct 12 located between a ballast tank 14 and a hull 16 of the shipping vessel 10. Analyzer system 100 may be positioned as close to the hull 16 as possible to ensure the highest quality measurement of the concentration of oxidants in the ballast water at the location of its highest likelihood of impact to the surrounding environment. Reagent 18 is shown schematically and may exist in any convenient location within the confines of the present invention. Suitable methods for mixing reagent 18 with the fluid sample are described in Applicant's U.S. Patent Application Publication No. 2016/0178594, entitled “Analyzer System and Method for Sensing a Chemical Characteristic of a Fluid Sample,” the entire disclosure of which is hereby incorporated herein by reference.

According to one embodiment as shown in FIG. 1, analyzer system 100 is mounted in a duct section 12a which is installed in-line as a modular component of discharge duct 12. Of course, those of ordinary skill in the art will appreciate that other mountings and/or locations of the analyzer system 100 are possible, as well, without departing from the spirit and scope of the present invention. In FIG. 1, process water flows from left to right, as designated by arrows 20.

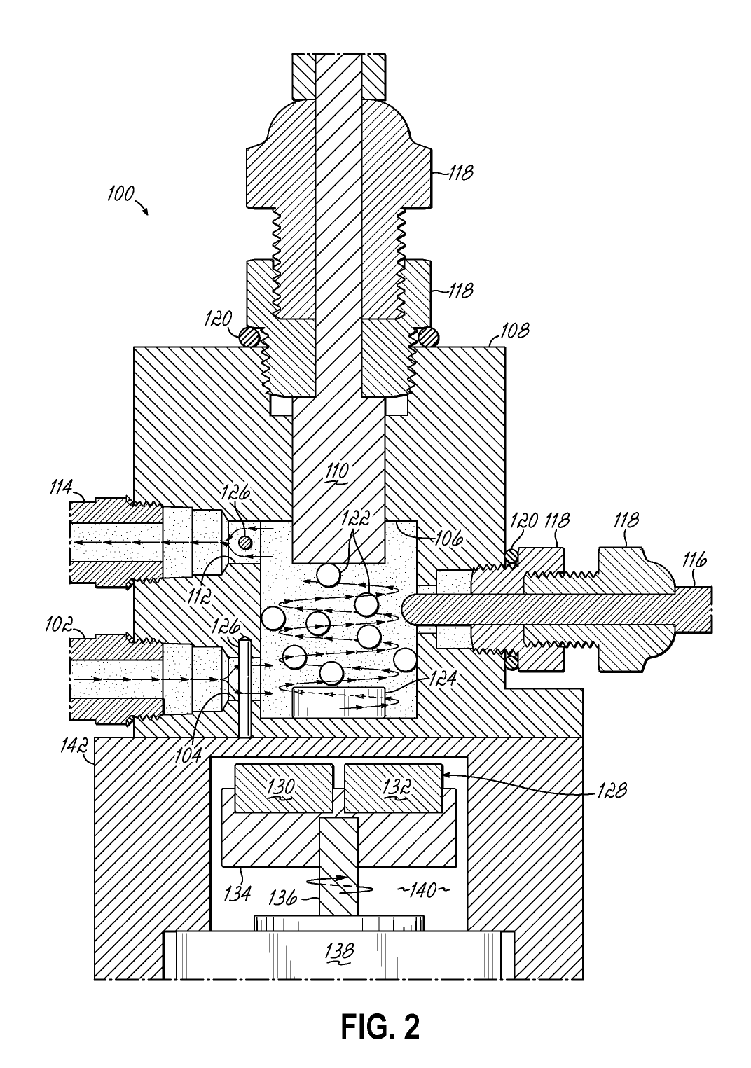

FIG. 2 illustrates the analyzer system 100 in greater detail, according to one embodiment of the present invention. As the flow of the process water approaches analyzer system 100, a small volume of process water, i.e. the sample, enters analyzer system 100 and is mixed with reagent 18. The treated fluid sample enters through an inlet channel 102 coupled to a sample inlet 104 and flows into a sensing chamber 106 within a housing 108. Sensing chamber 106 includes at least one sensor, such as oxidants probe 110, which may include, e.g., at least one sensing electrode and at least one reference electrode (not shown). The sensor may include an ion specific electrode. Suitable ion specific electrodes include, for example, Thermo Scientific™ Orion™ Industrial Ion Selective Electrodes, such as a chlorine sensing half-cell electrode (100020), and a Thermo Scientific™ Orion™ Chlorine Electrode (9770BNWP). The flow of the fluid sample carries it upwardly in a vertical direction toward oxidants probe 110, where a chemical characteristic of the process water sample is sensed, as will be discussed in greater detail below. The flow of process water in the discharge duct 12 pushes the fluid sample to sample outlet 112, which may be positioned vertically upwardly from sample inlet 104. Sample outlet 112 is fluidically coupled to outlet channel 114 through which the sample is expelled into discharge duct 12. The flow of process water through sensing chamber 106 may be continuous while the process water is flowing through discharge duct 12. Accordingly, oxidants probe 110 may be configured to sense the chemical characteristic of the fluid sample as it continuously flows through the sensing chamber 106. Sample outlet 112 is configured to prevent fluid flowing through discharge duct 12 from entering sensing chamber 106 (i.e., fluid does not move from discharge duct 12 into sample outlet 112). When the flow of process water in the discharge duct 12 ceases, at least a portion of the fluid present in the sensing chamber 106 will drain into discharge duct 12 via the sample inlet 104 and the fluid level will fall below the level of the oxidants probe 110. Optionally, one or more second probes 116 can be incorporated into sensing chamber 106. Second probe 116 may be, for example, a temperature probe or a pH probe. The position of second probe 116 may be off-center of the cell and with respect to stirrer 124. In an embodiment, electrode position spacing to sample outlet 112 is less than the diameter of cleaning beads 122. Suitable probes include, for example, a Thermo Scientific™ Orion™ Stainless-Steel Automatic Temperature Compensation (ATC) Probe (917007). Oxidants probe 110 and any optional second probes 116 may be coupled to housing 108, for example, using removable, interlocking fixtures 118 and O-rings 120 that create a fluid-tight seal.

To clean the surface of oxidants probe 110, sensing chamber 106 includes a plurality of cleaning beads 122 and an agitator, such as stirrer 124. Stirrer 124 is configured to mechanically stir the fluid sample in sensing chamber 106, which causes at least some of the plurality of cleaning beads 122 contact a surface of oxidants probe 110. In an embodiment including second probe 116, cleaning beads 122 may also clean the surface of second probe 116. Cleaning beads 122 should be capable of cleaning the surface of oxidants probe 110 without damaging it. For example, cleaning beads 122 may be made of glass, plastic, or rubber. In an embodiment, the cleaning beads 122 may have a rough surface, which aids in cleaning the surface of oxidants probe 110. While spherical cleaning beads 122 are shown in FIG. 1, cleaning beads 122 may have a shape that is an ovoid, curved in another manner, or a tetrahedron. Further, the position of each of oxidants probe 110 and optional second probe 116 may vary. The sensing surface of each probe or sensor may be exposed to impact by cleaning beads 122 at any angle, such as tangential or normal. In other words, each probe or sensor could be mounted vertically or horizontally with its sensing surface being in the center, off-center, or perpendicular to the rotation of cleaning beads 122. Additionally, sensing chamber 106 may include a volume of trapped air or gas that causes or increases foaming action when cleaning beads 122 are stirred. Foaming action helps trap waste, such as foam and dirt in the sample fluid, and also reduces the effective time when the sensing surface of oxidants probe 110 is in direct contact with liquid sample, while the tip was exposed to bombardment by cleaning beads 122.

Analyzer system 100 is configured to contain cleaning beads 122 within sensing chamber 106. In an embodiment, sample inlet and outlet 104, 112 each include barriers 126, such as pins or screws in registration with sample inlet and outlet 104, 112. Barriers 126 may be spaced a distance from the walls of the respective sample inlet and outlet 104, 112 that is at least half the size of the diameter of cleaning beads 122. This prevents cleaning beads 122 from escaping into discharge duct 12. In an embodiment, barrier 126 may be a mesh sized to prevent cleaning beads 122 from passing therethrough. The mesh may be cleaned by contact with cleaning beads 122 like oxidants probe 110.

In use, stirrer 124 will force cleaning beads 122 to start moving in a circulating motion contacting the surface of oxidants probe 110. This action of scrubbing the surface minimizes the accumulation of material on the surface. Contact between cleaning beads 122 cleans the surface of oxidants probe 110 and ensures that the oxidants probe 110 provides accurate readings. Thus, analyzer system 100 is self-cleaning due to the action of cleaning beads 122. Agitation of cleaning beads 122 also provides additional mixing of the fluid sample and reagent 18, which better homogenizes the fluid sample for sensing especially where the sample includes high levels of particulate matter (e.g., suspended solids). Further, the mechanical action of stirrer 124 allows for adjustable control of the movement of cleaning beads 122. Analyzer system 100 is not reliant on the flow rate of the fluid sample to ensure cleaning beads 122 make sufficient contact to effectively clean oxidants probe 110. Further, compared to motion of cleaning beads 122 due to the flow of the fluid sample alone, the motion of cleaning beads 122 is more uniform due to stirrer 124. In an aspect of the present invention, cleaning beads 122 may be directed to specific surfaces for cleaning.

Returning to the mechanical action of stirrer 124, in the illustrated embodiment, stirrer 124 is magnetic. Analyzer system 100 includes a rotating magnet 128 comprising opposite poles 130, 132 carried by a platform 134. Platform 134 is coupled to a rotating support 136 powered by a stepper motor 138. Rotating magnet 128, platform 134, and support 136 are contained in a sealed chamber 140 within a housing 142. Process fluid flowing through discharge duct 12 or sensing chamber 106 is not able to enter chamber 140. When support 136 rotates platform 134, the rotation of magnet 128 causes corresponding rotation of stirrer 124. Cleaning beads 122 should not interfere with the magnetic field between stirrer 124 and rotating magnet 128. Magnetic stirrer 124 may be in a form other than a stirrer bar. For example, stirrer 124 may include magnetic beads or a paddle. In an embodiment where stirrer 124 is magnetic, cleaning beads 122 may also be magnetic. For example, cleaning beads 122 may have an inner, magnetic core and an outer layer that is, for example, plastic, glass, or rubber.

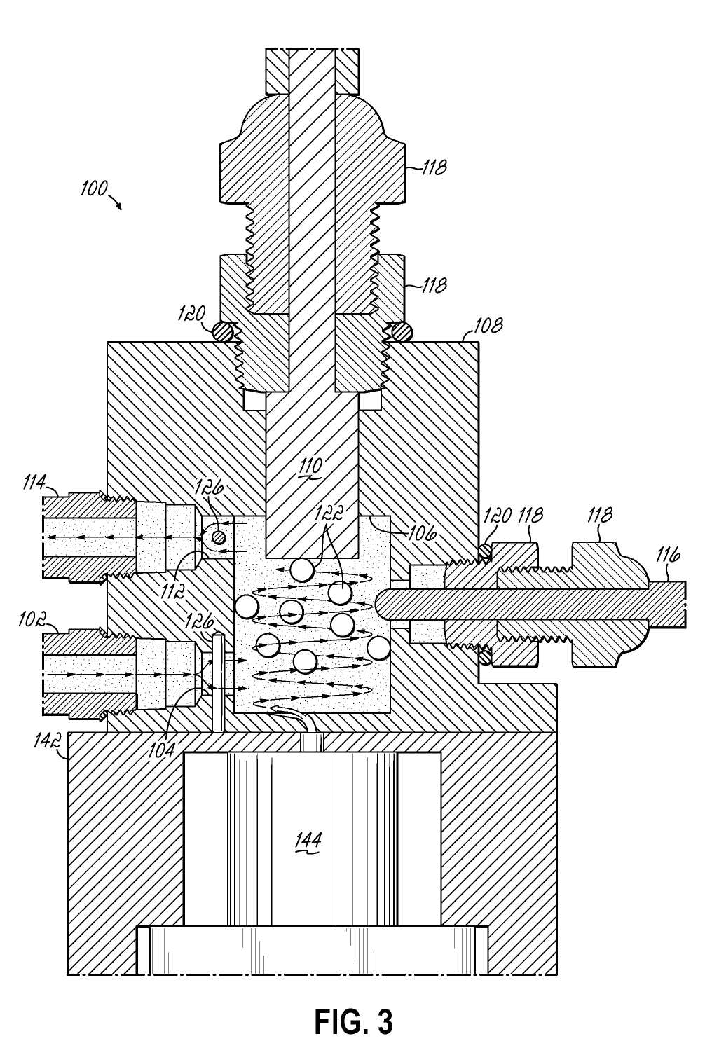

FIG. 3 illustrates an alternative agitator for use in an analyzer system, such as analyzer system 100, according to one embodiment of the present invention. In the illustrated embodiment, an air or gas source 144 is configured to agitate cleaning beads 122. A stream or pulse of air or gas is provided from source 144 to sensing chamber 106 to mechanically stir the fluid sample, which causes at least some of the plurality of cleaning beads 122 contact a surface of oxidants probe 110.

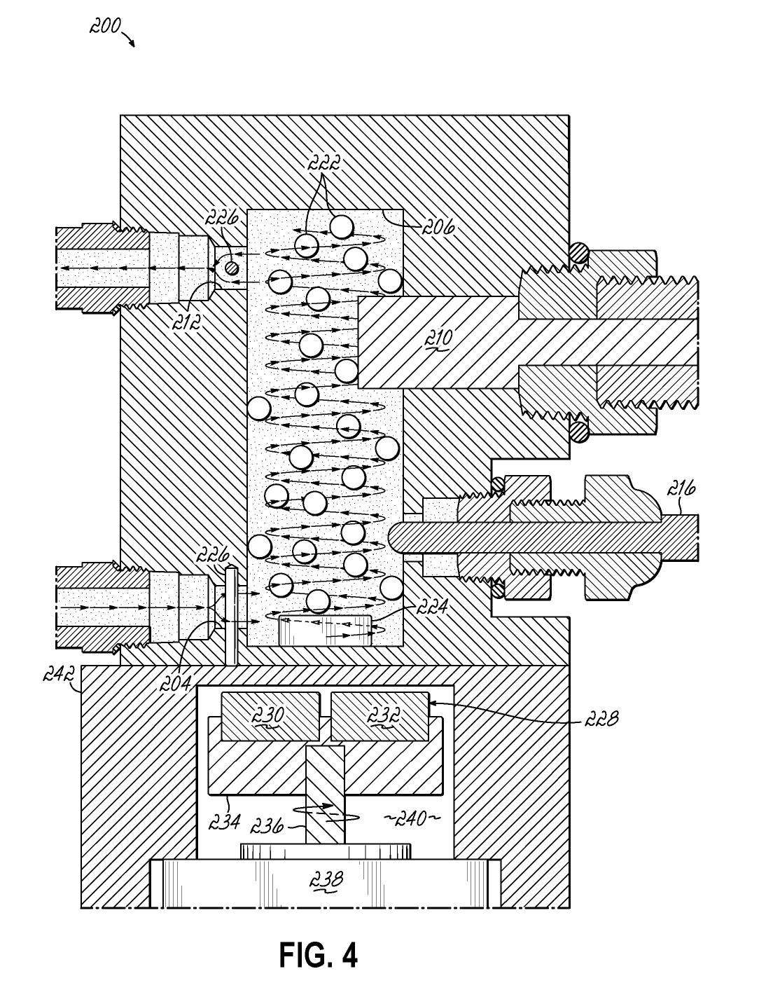

FIG. 4 illustrates a self-cleaning analyzer system 200 according to another embodiment of the present invention, shown mounted in the duct section 12a of discharge duct 12, in which the chemical characteristic of a fluid sample may be measured in a batch process. A batch sensing process may reduce the amount of reagent used during the sensing process. As the flow of the process water approaches analyzer system 200, a predetermined volume of process water, i.e. the sample, enters analyzer system 200. The fluid sample enters the sample inlet 204 and flows into a sensing chamber 206. Reagent 18 may be added to sensing chamber 206 or may be added to the sample before it enters sensing chamber 206. The sample and reagent 18 are held in sensing chamber 206 while the reagent 18 reacts with the sample. Sensing chamber 206 includes oxidants probe 210 and optional second probe 216. The oxidants probe 210 is positioned at a level between sample inlet and outlet 204, 212. The predetermined volume of the sample is enough to fill sensing chamber 206 to a level above oxidants probe 210 but below the level of sample outlet 212 so that the chemical characteristic of the sample may be measured without the sample flowing out of sample outlet 212.

To clean the surface of oxidants probe 210, sensing chamber 206 includes a plurality of cleaning beads 222 and a stirrer 224. Stirrer 224 is configured to stir the fluid sample in sensing chamber 206, which causes at least some of the plurality of cleaning beads 222 contact a surface of oxidants probe 210. Analyzer system 200 is configured to contain cleaning beads 222 within sensing chamber 206. As described above, in an embodiment, sample inlet and outlet 204, 212 each include barriers 226, such as pins or screws.

In use, stirrer 224 will force cleaning beads 222 to start moving in a circulating motion contacting the surface of oxidants probe 210. This action of scrubbing the surface minimizes the accumulation of material on the surface. Contact between the cleaning beads 222 cleans the surface of oxidants probe 210 and ensures that the oxidants probe 210 provides accurate readings. Thus, analyzer system 200 is self-cleaning due to the action of cleaning beads 222. Further, the mechanical action of stirrer 224 allows for adjustable control of the movement of cleaning beads 222. Analyzer system 200 is not reliant on the flow rate of the fluid sample to ensure cleaning beads 222 make sufficient contact to effectively clean oxidants probe 210. Further, compared to motion of cleaning beads 222 due to the flow of the sample alone, the motion of cleaning beads 222 is more uniform compared to motion directed by flowing sample. In an aspect of the present invention, cleaning beads 222 may be directed to specific surfaces for cleaning. Additionally, the movement of cleaning beads 222 mixes the sample and reagent 18 together, which may increase the speed and uniformity of the reaction in sensing chamber 206.

As illustrated, stirrer 224 is magnetic and may be controlled by rotating magnet 228. Rotating magnet 228, which comprises opposite poles 230, 232, platform 234, and rotating support 236 are contained in sealed chamber 240 within housing 242. When motor 238 rotates support 236, the rotation of magnet 228 causes corresponding rotation of stirrer 224.

After the fluid sample and reagent 18 have been mixed sufficiently and the chemical characteristic has been sensed, additional process fluid may be provided to sensing chamber 206 to flush the sensed fluid sample out of sensing chamber 206. Additionally, a cleaning agent may be provided to sensing chamber 206 after a fluid sample has been sensed.

As described above, the embodiment shown in FIGS. 2 and 3 are well-suited for continuous sensing process and the embodiment shown in FIG. 4 is well-suited for sensing in a batch process. However, the features described in each embodiment can easily be added to any of the alternative embodiments described in the present application. One of ordinary skill in the art is capable of modifying the analyzer systems 100, 200, within these general principles, to suit the requirements of the particular application.

In addition to the disclosed analyzer systems, the present invention also features a method for sensing a chemical characteristic of a fluid sample using a self-cleaning analyzer system. The method includes, for example, providing the fluid sample to the sensing chamber via the sample inlet; sensing the chemical characteristic of the fluid sample; and stirring the fluid sample via the agitator causing one or more of the plurality of cleaning beads to contact the sensor.

The sample is provided to the analyzer system through a sampling device capable of extracting a small sample flow rate from a large flow rate. Indeed, approximately a 1 million-fold reduction in flow rate is obtainable without additional power added to the system in the form of a pump or valve. In an embodiment of the invention, the sample inlet is a sample sipping apparatus within a process water duct. Sample sipping pertains to a design that withdraws a constant and known portion from a stream within a duct. In another embodiment, the sampling device may be capable of withdrawing a predetermined volume of the stream within the duct.

The reagent is provided to the analyzer system through any means capable of storing and delivering the appropriate amount of reagent for conducting the analysis. The term “reagent” may also include probe cleaning solution. The reagent may be a gas-phase (vapor), liquid, or solid, and its chemical composition depends upon the particular application and sensing approach used. For instance, TRO may be sensed using an iodometric approach with potassium iodide and acetic acid. Chlorine, phosphate, and silica, may be sensed using colorimetry with 2-(Diphosphonomethyl) succininc acid, vannado-molybdate, or molybdic acid, ascorbic acid, and heteropolyblue, respectively, for example. Potentiometric sensing may be used to monitor sodium, chloride, and fluoride, using diisopropylamine vapor or formic acid, for example. One of ordinary skill in the art is capable of selecting the appropriate sensing technique and associated reagent to monitor the chemical characteristic of interest, and the present invention is not intended to be limited to any particular sensing technique or reagent.

As explained above, sensing of TRO may be performed using iodometric techniques. An exemplary iodometric technique is described in U.S. Pat. No. 4,049,382, entitled “Total Residual Chlorine,” the entire disclosure of which is hereby incorporated herein by reference. Briefly, the sample stream is mixed with the reagent stream containing a dissociated complex of alkali metal ion and iodide ion, along with an excess amount of iodide ion. The iodide reacts with all residual chlorine in the sample and is converted to iodine. Two probes then measure the activity of the iodine, from which the total residual chlorine is determined.

In an embodiment, after providing the sample and reagent to the analyzer system, the reagent and the sample are mixed. Mixing can be accomplished, for example, by stirring the sample and reagent using the agitator and cleaning beads. Further, the sensing chamber may be configured in such a way that turbulence is created while the sample and reagent are flowing through the sensing chamber. In another embodiment, the reagent and sample are mixed before being provided to the sensing chamber.

The sensing chamber may be configured to allow continuous or batch process sensing of the chemical characteristic of the fluid sample using an ion specific sensor and other optional probes. For example, the fluid sample may flow continuously through the sensing chamber past the ion specific sensor and out the sample outlet. In an alternate embodiment, a predetermined volume of the fluid sample may be provided to the sensing chamber such that the chemical characteristic of the fluid sample may be sensed without the fluid sample exiting through the sample outlet. Once the chemical characteristic has been sensed, more process fluid or a cleaning agent may be provided to the sensing chamber to flush out the sensed fluid sample.

The analyzer system is configured to clean the surfaces of the ion specific sensor and other optional probes. In one embodiment, a motor is used to control a stirrer in the sensing chamber. The stirrer may be controlled magnetically. Rotation of the stirrer stirs the fluid sample and the plurality of cleaning beads. In another embodiment, a stream of air or gas is used to stir the fluid sample and cleaning beads. Due to the agitation, at least some of the cleaning beads contact the surface of the sensor(s), which prevents unwanted buildup on the surface. As described above, this configuration is not reliant on the flow rate of the fluid sample to ensure the cleaning beads make sufficient contact to effectively clean the surface of the sensor(s). This configuration also allows for batch sensing of the fluid sample without constant flow of the fluid sample into the sensing chamber. Further, the motion of the cleaning beads may be directed to specific surfaces for cleaning.

Additionally, as described above, the sensed fluid sample is returned to the process water duct, which is a feasible solution to waste-stream generation when the reagent is not particularly hazardous. However, the invention is not limited to only such waste reinjection. In situations where a hazardous material, such as chromium or mercury, is used as the reagent, one of ordinary skill in the art is capable of modifying the embodiments shown to allow for collection of a waste stream to hold for proper disposal.

An analyzer system according to an embodiment of the present invention was made and field tested. The following sizes and configuration are provided as an example but do not limit the invention. Tubing with a diameter of 4 mm was used for the inlet and outlet channels. The inlet and outlet channels were coupled to the sample inlet and outlet, respectively, which each had a diameter of 6 mm. The diameter of the cleaning beads was 3 mm, and the width of the barriers was 2 mm. The sensing chamber was 37 mm in diameter and 20 mm deep. The effective volume of the sensing chamber was 9.8 mm. A stepper motor with a 5 mm shaft was used to rotate the rotating magnet, which in turn caused the magnetic stirrer to rotate. Clockwise motion of the stirrer and cleaning beads brought the sample in and straight across the surface of the sensor (12 mm diameter). The cleaning beads both mixed the sample with the reagent and cleaned the surface of the sensor.

While the various principles of the invention have been illustrated by way of describing various exemplary embodiments, and while such embodiments have been described in considerable detail, there is no intention to restrict, or in any way limit, the scope of the appended claims to such detail. Additional advantages and modifications will readily appear to those skilled in the art.

As various changes could be made in the above-described aspects and exemplary embodiments without departing from the scope of the invention, it is intended that all matter contained in the above description shall be interpreted as illustrative and not in a limiting sense.

What is claimed is:

1. A self-cleaning analyzer system for sensing a chemical characteristic of a fluid sample, comprising:
a sensing chamber including a sample inlet configured to receive the fluid sample and a sample outlet;
a sensor configured to sense the chemical characteristic of the fluid sample in the sensing chamber;
a plurality of cleaning beads contained in the sensing chamber; and
an agitator configured to stir the fluid sample and the plurality of cleaning beads in the sensing chamber,
wherein one or more of the plurality of cleaning beads contact the sensor when the agitator stirs the fluid sample.

2. The system of claim 1, wherein the agitator is configured to achieve homogenous mixing of the fluid sample and a reagent.

3. The system of claim 1, wherein the sensing chamber includes a volume of trapped air or gas for trapping waste.

4. The system of claim 1, wherein the agitator is a stirrer.

5. The system of claim 4, wherein the stirrer is magnetic, the system further comprising a rotating magnet configured to cause rotation of the stirrer.

6. The system of claim 1, wherein the agitator is a stream or pulse of air or gas.

7. The system of claim 1, wherein the sample inlet and sample outlet are configured to prevent movement of the cleaning beads outside of the sensing chamber.

8. The system of claim 7, wherein the sample inlet and sample outlet include one or more pins in registration with the sample inlet and sample outlet, respectively, that are sized to prevent the cleaning beads from entering the sample inlet and sample outlet.

9. The system of claim 7, wherein the sample inlet and sample outlet include a mesh in registration with the sample inlet and sample outlet that is sized to prevent the cleaning beads from entering the sample inlet and sample outlet.

10. The system of claim 1, wherein the cleaning beads are balls with a diameter of about 3 mm.

11. The system of claim 1, wherein the cleaning beads are glass balls.

12. The system of claim 1, wherein the cleaning beads are plastic balls.

13. The system of claim 1, further comprising a temperature probe configured to sense a temperature of the fluid sample in the sensing chamber.

14. The system of claim 1, wherein the sensor is configured to sense the chemical characteristic of the fluid sample as it continuously flows through the sensing chamber.

15. The system of claim 1, wherein the sample outlet is positioned at a height above the sample inlet and sensor to allow for a batch sensing process.

16. The system of claim 1, wherein the chemical characteristic comprises a total residual oxidants present in the fluid sample.

17. The system of claim 1, wherein the system is a component of a ballast water pipe.

18. A method for sensing a chemical characteristic of a fluid sample using the self-cleaning analyzer system of claim 1, comprising:
providing the fluid sample to the sensing chamber via the sample inlet;
sensing the chemical characteristic of the fluid sample; and
stirring the fluid sample and the cleaning beads in the sensing chamber via the agitator causing one or more of the plurality of cleaning beads to contact the sensor.

19. The method of claim 18, wherein providing the fluid sample to the analyzer system is continuous.

20. The method of claim 18, wherein providing the fluid sample to the sensing chamber comprises providing a predetermined volume of the fluid sample and sensing the chemical characteristic of the fluid sample is a batch process.

21. The method of claim 18, further comprising mixing a reagent with the fluid sample.

22. The method of claim 21, wherein mixing the reagent with the fluid sample occurs prior to providing the fluid sample to the sensing chamber.

23. The method of claim 21, wherein mixing the reagent with the fluid sample occurs after providing the fluid sample to the sensing chamber.
